(19) United States
(12) Patent Application Publication
(10) Pub. No.: US 2009/0068271 A1
(43) Pub. Date: Mar. 12, 2009

(54) EMBOLIZATION PARTICLES

(75) Inventors: Paul DiCarlo, Middleboro, MA (US); Barron W. Tenney, Haverhill, MA (US); Yixin Xu, Newton Lower Falls, MA (US); Michael N. Helmus, Worcester, MA (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 12/179,115

(22) Filed: Jul. 24, 2008

Related U.S. Application Data
(60) Provisional application No. 60/971,705, filed on Sep. 12, 2007.

(51) Int. Cl. A61K 9/14; B32B 15/02; B32B 27/02

(52) U.S. Cl. 424/489; 428/401; 428/375; 428/379; 428/377; 428/376

(57) ABSTRACT

Embolic particles, embolic particle chains, and methods for making embolic particles and embolic particle chains are described.

### CROSS-REFERENCE TO RELATED APPLICATION

This application claims priority under 35 U.S.C. §119 to U.S. Ser. No. 60/971,705, filed Sep. 12, 2007, the contents of which are hereby incorporated by reference.

### FIELD

The technology described herein relates to particles and methods for making particles that are used for embolization.

### BACKGROUND

Embolic particles can be used to create therapeutic vascular occlusions, which are used to prevent or to treat certain pathological conditions in the body. For example, in therapeutic vascular occlusions (sometimes called “embolizations”), embolic particle compositions can be used to block, or occlude, vessels in the body. As further examples, embolic particle compositions can be used to block microvascular supplies of blood to tumors (thereby depriving the tumors of resources to grow), or to block hemorrhagic conditions in the body (thereby reducing or stopping bleeding). The compositions can be delivered to a target site using a catheter.

### SUMMARY

Embolic particles, embolic particle chains, and methods for making embolic particles and embolic particle chains are described herein.

In one aspect, the particles described herein include one or more polymer fiber strands that are collectively organized into the overall shape of a particle. In this particle, the polymer fiber strands are distributed such that voids are formed within the particle.

In another aspect, a particle as described herein can be connected by a link to another particle.

In a further aspect, a method for forming a particle is described. In this method, a polymer strand is disposed into a mold to form a particle. The largest dimension of the mold is at most 5,000 microns.

In another aspect, another method for forming a particle is described. In this method, an electrostatically charged polymer fiber strand is disposed into a mold with an electrostatically charged interior surface to form the particle. The charged fiber strand is electrostatically attracted to the interior surface of the mold.

In an additional aspect, a method is described in which an electrostatically charged polymer is directed from a nozzle toward an electrode. The electrostatically charged polymer in this method has a charge opposite the electrode. With this method a chain is formed of at least two particles linked by a filament.

### BRIEF DESCRIPTION OF THE FIGURES

FIG. 1A is an illustration of an embodiment of an embolic particle.

FIG. 1B is a cross-sectional view at line 1B of an embodiment of an embolic particle as shown in FIG. 1A.

FIG. 1C is a cross-sectional view of an embodiment of an embolic particle that includes a core.

FIG. 1D is an illustration of an embodiment of a particle chain.

FIG. 2 is a illustration of an embodiment of a system used to manufacture an embolic particle.

FIG. 3 is a illustration of an embodiment of a system used to manufacture an embolic particle chain.

FIG. 4A is a schematic illustrating an embodiment of injection of an embolic composition into a vessel, and FIG. 4B is an enlarged view of the region 4B in FIG. 4A.

FIG. 5 is an illustration of an embodiment of an embolic particle that includes a coating.

### DETAILED DESCRIPTION

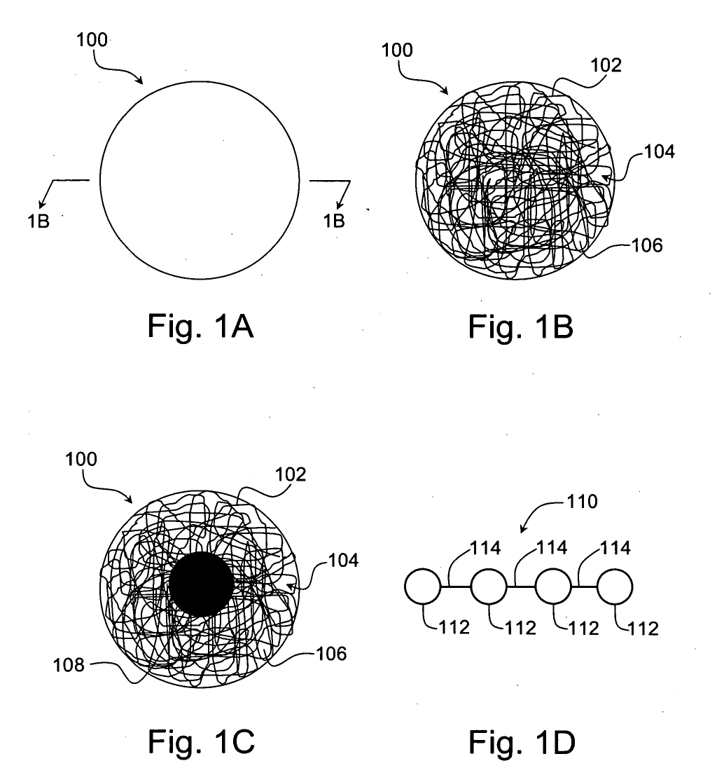

FIGS. 1A, 1B, and 1C show a particle 100 that can be used, for example, to deliver one or more therapeutic agents to a target site within the body. The particle 100 is formed from polymer fiber strands 102 that are collectively organized into the overall shape of a particle 100. These particle fiber strands 102 together form a particle fiber matrix 104 that includes voids 106. The term “voids” is intended to mean the spaces between polymer fiber strands within a particle fiber matrix. The polymer fiber strands 102 can be randomly oriented (i. e., without a recurring or overall pattern) or, alternatively, the polymer fiber strands 102 in the particle fiber matrix 104 can be organized in an overall pattern (or recurring pattern). Therapeutic agent(s) can be included on and/or within particle 100 (e.g., within the polymer fiber strands 102 and/or within the voids 106 of the particle fiber matrix 104).

The particle 100 can be formed from a single polymer fiber strand or from multiple strands. In multiple strand embodiments, the individual strands can be formed from the same polymer or different polymers. The particle fiber strands 102 of particle 100 can be formed of a homogeneous polymer or a block copolymer. Each fiber strand can be physically bonded at one or more points along its length to an overlapping intra-strand or inter-strand segment.

The polymer fiber strands 102 can be elastic. The term “elastic” as used herein is intended to mean capable of being stretched or expanded when a force is applied while retaining the ability to resume or substantially resume the pre-stretched or pre-expanded shape when the force is removed. In some embodiments the particle 100 is compressible. The term “compressible” as used herein is intended to mean capable of being reduced or altered in size or volume when a force is applied while retaining the ability to resume or substantially resume the pre-reduced or pre-altered shape when the force is removed.

In some embodiments, the voids 106 within the particle fiber matrix 104 can be shielded, i.e., not directly accessible from the exterior of the particle 100. In certain embodiments, the voids 106 within the particle fiber matrix 104 can also be directly accessible from the exterior of the particle. Optionally, in a given particle 100, the voids 106 can be both shielded and accessible.

As shown in FIG. 1C, in a further embodiment, the particle 100 can include a core 108. The core 108 can be a polymer or a metal. The core 108 can be the same material as the polymer fiber strands 102 or a different material. The core 108 can be electrically charged. The core 108 can be a radioactive material. The core 108 can be hollow. The polymer fiber strands 102 can be wound, i.e., wrapped, around the core to form the overall shape of the particle.

In general, the largest dimension of particle 100 is 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of particle 100 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of particle 100 is less than 100 microns (e.g., less than 50 microns).

In some embodiments, the particle 100 can be substantially spherical. In certain embodiments, the particle 100 can have a sphericity of about 0.8 or more (e.g., about 0.85 or more, about 0.9 or more, about 0.95 or more, about 0.97 or more). For embodiments in which particle 100 is compressible, the particle 100 can be, for example, manually compressed (flattened) while wet to about 50 percent or less of its original largest dimension and then, upon exposure to fluid, regain a sphericity of about 0.8 or more (e.g., about 0.85 or more, about 0.9 or more, about 0.95 or more, about 0.97 or more). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=√(4A/π); Dp=P/π; A=pixel area; P=pixel perimeter), is a value from zero to one, with one representing a perfect circle.

In some embodiments, two or more particles 100 can be linked together to form a particle chain 110 as shown in FIG. 1D, e.g., a particle portion 112 of the particle chain 110 can be connected by a linkage portion 114 to at least one other particle portion 112. The particle portions 112 can be connected to each other in the particle chain 110 by linkage portions 114 that are formed of one or more of the same material(s) as the particle portions 112, or of one or more different material(s) from the particle portions 112. For example, the linkage portions 114 can be formed from a polymer, a metal, or a fiber. Additionally, a particle portion 112 can be connected to a particle or particles dissimilar to particle portion 112.

In general, a particle portion 112 can have a largest dimension of 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of particle portion 112 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of particle portion 112 is less than 100 microns (e.g., less than 50 microns).

In some embodiments, a particle portion 112 can be substantially spherical. In certain embodiments, a particle portion 112 can have a sphericity of about 0.8 or more (e.g., about 0.85 or more, about 0.9 or more, about 0.95 or more, about 0.97 or more). In some embodiments, the particle portion 112 is compressible. The particle portion 112 can be, for example, manually compressed, essentially flattened, while wet to about 50 percent or less of its original largest dimension and then, upon exposure to fluid, regain a sphericity of about 0.8 or more (e.g., about 0.85 or more, about 0.9 or more, about 0.95 or more, about 0.97 or more). The sphericity of a particle can be determined using a Beckman Coulter RapidVUE Image Analyzer version 2.06 (Beckman Coulter, Miami, Fla.). Briefly, the RapidVUE takes an image of continuous-tone (gray-scale) form and converts it to a digital form through the process of sampling and quantization. The system software identifies and measures particles in an image in the form of a fiber, rod or sphere. The sphericity of a particle, which is computed as Da/Dp (where Da=i(4A/7r); Dp=P/7r ; A=pixel area; P=pixel perimeter), is a value from zero to one, with one representing a perfect circle.

In general, the particle chain 110 can have a restrained length of from about one centimeter to about 50 centimeters. The restrained length LR of the particle chain 110 is the maximum length of the particle chain 110 (the length of the particle chain 110 when the particle chain 110 is taut) in any dimension. In some embodiments, the particle chain 110 can have a restrained length of at least about one centimeter (e.g., at least about five centimeters, at least about ten centimeters, at least about 15 centimeters, at least about 20 centimeters, at least about 25 centimeters, at least about 30 centimeters, at least about 35 centimeters, at least about 40 centimeters, at least about 45 centimeters) and/or at most about 50 centimeters (e.g., at most about 45 centimeters, at most about 40 centimeters, at most about 35 centimeters, at most about 30 centimeters, at most about 25 centimeters, at most about 20 centimeters, at most about 15 centimeters, at most about ten centimeters, at most about five centimeters).

The particle chain 110 includes at least two particle portions 112 (e.g., from two particle portions to 1,000 particle portions). In some embodiments, the particle chain 110 can include at least two particle portions 112 (e.g., at least five particle portions; at least ten particle portions; at least 20 particle portions; at least 30 particle portions; at least 40 particle portions; at least 50 particle portions; at least 100 particle portions; at least 250 particle portions; at least 500 particle portions; at least 750 particle portions; at least 1,000 particle portions; at least 2,500 particle portions) and/or at most 5,000 particle portions (e.g., at most 2,500 particle portions; at most 1,000 particle portions; at most 750 particle portions; at most 500 particle portions; at most 250 particle portions; at most 100 particle portions; at most 50 particle portions; at most 40 particle portions; at most 30 particle portions; at most 20 particle portions; at most ten particle portions; at most five particle portions). For example, the particle chain 110 can include five particle portions, ten particle portions, 100 particle portions, 500 particle portions, or 1,000 particle portions.

The particle portions 112 in the particle chain 110 can all have approximately the same largest dimension or can have different largest dimension. As an example, in some embodiments, the particle portions 112 at one end of the particle chain 110 can have a larger largest dimension (e.g., by about 1100 microns) than the particle portions 112 at the other end of the particle chain 110. As another example, in certain embodiments, the particle portions 112 in the particle chain 110 can alternate in size. For example, a particle portion 112 with a largest dimension of about 300 microns can be adjacent to a particle portion 112 with a largest dimension of about 500 microns.

The linkage portions 114 generally can have a width of from 0.001 inch to about 0.01 inch (e.g., from 0.003 inch to 0.005 inch). In certain embodiments, the linkage portions 114 can have a width of at least 0.001 inch (e.g., at least 0.002 inch, at least 0.003 inch, at least 0.004 inch, at least 0.005 inch, at least 0.006 inch, at least 0.007 inch, at least 0.008 inch, at least 0.009 inch) and/or at most about 0.01 inch (e.g., at most 0.009 inch, at most 0.008 inch, at most 0.007 inch, at most 0.006 inch, at most 0.005 inch, at most 0.004 inch, at most 0.003 inch, at most 0.002 inch).

In some embodiments, the linkage portions 114 in the particle portion 112 can all have approximately the same length and/or width. In other embodiments, the particle portion 112 can include linkage portions 114 of varying lengths and/or widths. As an example, in certain embodiments, one end of a particle chain 110 can have relatively short, thick links, while the other end of the particle chain 110 has relatively long, thin links. As another example, in some embodiments, the linkage portions 114 in a particle chain 110 can alternate between being relatively short and thick and relatively long and thin.

In general, the linkage portions 114 can have an aspect ratio (the ratio of the length of the link to the width of the link) of from about zero to about 1,000. In some embodiments, the linkage portions 114 can have an aspect ratio of at least 0.001 (e.g., at least 0.005, at least about 0.5, at least about one, at least about five, at least about ten, at least about 15, at least about 20, at least about 25, at least about 26, at least about 30, at least about 40, at least about 50, at least about 75, at least about 100, at least about 200, at least about 300, at least about 400, at least about 500, at least about 600, at least about 700, at least about 800, at least about 900 ) and/or at most about 1,000 (e.g., at most about 900, at most about 800, at most about 700, at most about 600, at most about 500, at most about 400, at most about 300, at most about 200, at most about 100, at most about 75, at most about 50, at most about 40, at most about 30, at most about 26, at most about 25, at most about 20, at most about 15, at most about ten, at most about five, at most about one, at most about 0.5, at most 0.005).

In general, the aspect ratio of the linkage portions 114 can be varied as desired. Typically, as the aspect ratio of the linkage portions 114 increases, the flexibility of the linkage portions 114 increases. As the aspect ratio of the linkage portions 114 decreases, the tensile strength of the linkage portions 114 typically increases.

In some embodiments, the ratio of the largest dimension of a particle portion 112 to the width of a linkage portions 114 can be from about 0.5 to about 100. The ratio can be at least about 0.5 (e.g., at least about 0.8, at least about one, at least about two, at least about five, at least about ten, at least about 12, at least about 15, at least about 20, at least about 25, at least about 30, at least about 35, at least about 40, at least about 45, at least about 50, at least about 55, at least about 60, at least about 70, at least about 80, at least about 90) and/or at most about 100 (e.g., at most about 90, at most about 80, at most about 70, at most about 60, at most about 55, at most about 50, at most about 45, at most about 40, at most about 35, at most about 30, at most about 25, at most about 20, at most about 15, at most about 12, at most about ten, at most about five, at most about two, at most about one, at most about 0.8).

Generally, the ratio of the largest dimension of a particle portion 112 to the width of a linkage portions 114 can be varied as desired. Typically, as the ratio of the largest dimension of a particle portion 112 to the width of a linkage portions 114 increases, the flexibility of the linkage portions 114 increases. As the ratio of the largest dimension of a particle portion 112 to the width of a linkage portion decreases, the tensile strength of the linkage portions 114 typically increases.

Particle chains and their characteristics are further described, for example, in Buiser et al., U.S. Patent Application Publication No. US 2005/0238870 A1, published on Oct. 27, 2005, and entitled “Embolization,” which is incorporated herein by reference.

Polymers useful in the particles described herein include homopolymers and copolymers. The term “homopolymer” as used herein refers to a polymer formed from identical monomer subunits. The term “copolymer” as used herein refers to a polymer formed from two or more monomer subunits. Examples of homopolymers useful in the particles described herein include, but are not limited to, polyvinyl alcohols (“PVA”), polyacrylic acids, polymethacrylic acids, poly vinyl sulfonates, carboxymethyl celluloses, hydroxyethyl celluloses, substituted celluloses, polyacrylamides, polyethylene glycols, polyamides, polyureas, polyurethanes, polyesters, polyethers, polystyrenes, polysaccharides, polylactic acids, polyethylenes, polyolefins, polypropylenes, polymethylmethacrylates, polycaprolactones, polyglycolic acids, poly(lactic-co-glycolic) acids (e.g., poly(d-lactic-co-glycolic) acids), polysulfones, polyethersulfones, polycarbonates, nylons, silicones, and linear or crosslinked polysilicones. Copolymers useful with the particles described herein can be formed from combinations of the monomers that make up these homopolymers.

Block copolymers are also useful with the particles described herein. The term “block copolymer” as used herein refers to copolymers that contain two or more differing polymer blocks selected, for example, from homopolymer blocks, copolymer blocks (e.g., random copolymer blocks, statistical copolymer blocks, gradient copolymer blocks, periodic copolymer blocks), and combinations of homopolymer and copolymer blocks. A polymer “block” refers to a grouping of multiple copies of a single type (homopolymer block) or multiple types (copolymer block) of constitutional units. A “chain” is an unbranched polymer block. In some embodiments, a polymer block can be a grouping of at least two (e.g., at least five, at least 10, at least 20, at least 50, at least 100, at least 250, at least 500, at least 750) and/or at most 1000 (e.g., at most 750, at most 500, at most 250, at most 100, at most 50, at most 20, at most 10, at most five) copies of a single type or multiple types of constitutional units. A polymer block may take on any of a number of different architectures.

In some embodiments, the block copolymer useful with the particles described herein can include a central block having a glass transition temperature of at most 37° C. and end blocks each having a glass transition temperature of greater than 37° C. In certain embodiments, the block copolymer can have one of the following general structures:
(a) BAB or ABA (linear triblock),
(b) B(AB)ₙ or A(BA)ₙ (linear alternating block), or
(c) X-(AB)ₙ or X-(BA)ₙ (includes diblock, triblock and other radial block copolymers),
   where A is a block having a glass transition temperature of at most 37° C., B is a block having a glass transition temperature of greater than 37° C., n is a positive whole number and X is an initiator (e.g., a monofunctional initiator, a multifunctional initiator).

The X-(AB)ₙ structures are frequently referred to as diblock copolymers (when n=1) or triblock copolymers (when n=2). (This terminology disregards the presence of the initiator, for example, treating A-X-A as a single A block with the triblock therefore denoted as BAB.) Where n=3 or more, these structures are commonly referred to as star-shaped block copolymers.

As described above, the A blocks have a glass transition temperature of at most 37° C. In some embodiments, the A blocks can have a glass transition temperature of at most about 30° C. (e.g., at most about 25° C., at most about 20° C, at most about 10° C., at most about 0° C., at most about −10° C., at most about −20° C., at most about −30° C., at most about −50° C., at most about −70° C., at most about −90° C.). As referred to herein, the glass transition temperature of a material (e.g., a polymer block) is determined according to ASTM E1356. Examples of blocks having a glass transition temperature of at most 37° C. when the blocks are in the dry state (e.g., in powder form) include blocks including at least one of the following monomers:
(1) acrylic monomers including:
   (a) alkyl acrylates, such as methyl acrylate, ethyl acrylate, propyl acrylate, isopropyl acrylate (e.g., isotactic isopropyl acrylate), butyl acrylate, sec-butyl acrylate, isobutyl acrylate, cyclohexyl acrylate, 2-ethylhexyl acrylate, dodecyl acrylate and hexadecyl acrylate,
   (b) arylalkyl acrylates, such as benzyl acrylate,
   (c) alkoxyalkyl acrylates, such as 2-ethoxyethyl acrylate and 2-methoxyethyl acrylate,
   (d) halo-alkyl acrylates, such as 2,2,2-trifluoroethyl acrylate, and
   (e) cyano-alkyl acrylates, such as 2-cyanoethyl acrylate;
(2) methacrylic monomers including:
   (a) alkyl methacrylates, such as butyl methacrylate, hexyl methacrylate, 2-ethylhexyl methacrylate, octyl methacrylate, dodecyl methacrylate, hexadecyl methacrylate and octadecyl methacrylate, and
   (b) aminoalkyl methacrylates, such as diethylaminoethyl methacrylate and 2-tert-butyl-aminoethyl methacrylate;
(3) vinyl ether monomers including:
   (a) alkyl vinyl ethers, such as methyl vinyl ether, ethyl vinyl ether, propyl vinyl ether, butyl vinyl ether, isobutyl vinyl ether, 2-ethylhexyl vinyl ether and dodecyl vinyl ether;
(4) cyclic ether monomers, such as tetrahydrofuran, trimethylene oxide, ethylene oxide, propylene oxide, methyl glycidyl ether, butyl glycidyl ether, allyl glycidyl ether, epibromohydrin, epichlorohydrin, 1,2-epoxybutane, 1,2-epoxyoctane, and 1,2-epoxydecane;
(5) ester monomers (other than acrylates and methacrylates), such as ethylene malonate, vinyl acetate, and vinyl propionate;
(6) alkene monomers, such as ethylene, propylene, isobutylene, 1-butene, trans-butadiene, 4-methyl pentene, 1-octene and other α-olefins, cis-isoprene, and trans-isoprene;
(7) halogenated alkene monomers, such as vinylidene chloride, vinylidene fluoride, cis-chlorobutadiene, and trans-chlorobutadiene;
(8) siloxane monomers, such as dimethylsiloxane, diethylsiloxane, methylethylsiloxane, methylphenylsiloxane, and diphenylsiloxane; and
(9) maleic monomers, such as maleic anhydride.

In certain embodiments, the A blocks can include one or more derivatives of the above monomers.

In some embodiments, the A blocks can be based upon one or more polyolefins. In certain embodiments, the A blocks can be polyolefinic blocks having alternating quaternary and secondary carbons of the general formulation: —(CRR′—CH₂)ₙ—, where R and R′ are linear or branched aliphatic groups (e.g., methyl, ethyl, propyl, isopropyl, butyl, isobutyl) or cyclic aliphatic groups (e.g., cyclohexane, cyclopentane), with and without pendant groups. For example, the A blocks can be polyolefinic blocks having the above formula, in which R and R′ are the same. As an example, the A blocks can be based on isobutylene:

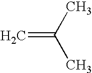

(i.e., in which R and R′ are both methyl groups).

In some embodiments, the block copolymer can include at least about 40 mol percent (e.g., from about 45 mol percent to about 95 mol percent) of polyolefin blocks.

As described above, the B blocks have a glass transition temperature of greater than 37° C. In some embodiments, the B blocks can have a glass transition temperature of at least about 40° C. (e.g., at least about 50° C., at least about 70° C., at least about 90° C., at least a 100° C., at least about 120° C.). Examples of blocks having a glass transition temperature of greater than 37° C. when the blocks are in the dry state (e.g., in powder form) include blocks including at least one of the following monomers:
(1) vinyl aromatic monomers including:
   (a) unsubstituted vinyl aromatics, such as atactic styrene, isotactic styrene and 2-vinyl naphthalene,
   (b) vinyl-substituted aromatics, such as α-methyl styrene, and
   (c) ring-substituted vinyl aromatics including ring-alkylated vinyl aromatics (e.g., 3-methylstyrene, 4-methylstyrene, 2,4-dimethylstyrene, 2,5-dimethylstyrene, 3,5-dimethylstyrene, 2,4,6-trimethylstyrene, 4-tert-butylstyrene), ring-alkoxylated vinyl aromatics (e.g., 4-methoxystyrene, 4-ethoxystyrene), ring-halogenated vinyl aromatics (e.g., 2-chlorostyrene, 3-chlorostyrene, 4-chlorostyrene, 2,6-dichlorostyrene, 4-bromostyrene, 4-fluorostyrene), ring-ester-substituted vinyl aromatics (e.g., 4-acetoxystyrene), and hydroxyl styrene;
(2) other vinyl monomers including:
   (a) vinyl esters such as vinyl benzoate, vinyl 4-tert-butyl benzoate, vinyl cyclohexanoate, vinyl pivalate, vinyl trifluoroacetate, vinyl butyral,
   (b) vinyl amines such as 2-vinyl pyridine, 4-vinyl pyridine, and vinyl carbazole,
   (c) vinyl halides such as vinyl chloride and vinyl fluoride,
   (d) alkyl vinyl ethers such as tert-butyl vinyl ether and cyclohexyl vinyl ether, and
   (e) other vinyl compounds such as vinyl ferrocene;
(3) other aromatic monomers including acenaphthalene and indene;
(4) methacrylic monomers including:
   (a) methacrylic acid anhydride,
   (b) methacrylic acid esters (methacrylates) including
      (i) alkyl methacrylates such as atactic methyl methacrylate, syndiotactic methyl methacrylate, ethyl methacrylate, isopropyl methacrylate, isobutyl methacrylate, t-butyl methacrylate and cyclohexyl methacrylate,
      (ii) aromatic methacrylates such as phenyl methacrylate and including aromatic alkyl methacrylates such as benzyl methacrylate,
      (iii) hydroxyalkyl methacrylates such as 2-hydroxyethyl methacrylate and 2-hydroxypropyl methacrylate,
      (iv) additional methacrylates including isobornyl methacrylate and trimethylsilyl methacrylate, and
   (c) other methacrylic-acid derivatives including methacrylonitrile;
(5) acrylic monomers including:
   (a) certain acrylic acid esters such as tert-butyl acrylate, hexyl acrylate and isobornyl acrylate,
   (b) other acrylic-acid derivatives including acrylonitrile; and
(6) silicate monomers including polyhedral oligomeric silsesquioxane (POSS) monomers.

In some embodiments, the B blocks can include one or more derivatives of the above monomers.

In certain embodiments, the B blocks can be polymers of methacrylates or polymers of vinyl aromatics. In some embodiments, the B blocks can be either: (a) made from monomers of styrene:

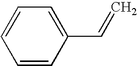

or styrene derivatives (e.g., α-methylstyrene, ring-alkylated styrenes or ring-halogenated styrenes) or mixtures thereof, or (b) made from monomers of methylmethacrylate, ethylmethacrylate, hydroxyethyl methacrylate, or mixtures thereof.

In some embodiments, the block copolymer can include at least about five mol percent (e.g., at least about 30 mol percent, about 60 mol percent) of styrene blocks.

An example of one of the above copolymers is styrene-isobutylene-styrene (“SIBS”), in which the A blocks are based on isobutylene, and the B blocks are based on styrene. Another example of one of the above copolymers is styrene maleic anhydride (“SMA”), in which the A blocks are based on maleic anhydride and the B blocks are based on styrene.

Typically, the combined molecular weight of the block copolymer can be more than about 40,000 Daltons (e.g., more than about 60,000 Daltons). For example, the combined molecular weight of the block copolymer can be from about 80,000 Daltons to about 300,000 Daltons (e.g., from about 90,000 Daltons to about 300,000 Daltons). In some embodiments (e.g., embodiments in which the A blocks are polyolefin blocks), the combined molecular weight of the A blocks can be from about 60,000 Daltons to about 200,000 Daltons. In certain embodiments (e.g., embodiments in which the B blocks are vinyl aromatic blocks), the combined molecular weight of the B blocks can be from about 20,000 Daltons to about 100,000 Daltons.

Generally, the properties of the block copolymer used in particle 100 can depend upon the lengths of the A block chains and B block chains in the block copolymer, and/or on the relative amounts of A block and B blocks in the block copolymer.

As an example, in some embodiments, blocks with a glass transition temperature of at most 37° C. may be elastomeric. In such embodiments, the elastomeric properties of the block copolymer can depend on the length of the A block chains. In certain embodiments, the A block chains can have a weight average molecular weight of from about 2,000 Daltons to about 30,000 Daltons. In such embodiments, the block copolymer and/or particle 100 may be relatively inelastic. In some embodiments, the A block chains can have a weight average molecular weight of at least about 40,000 Daltons. In such embodiments, the block copolymer and/or particle 100 may be relatively soft and/or rubbery.

As another example, in certain embodiments, blocks with a glass transition temperature of greater than 37° C. may be relatively hard at 37° C. In such embodiments, the hardness of the block copolymer at 37° C. can depend on the relative amount of B blocks in the block copolymer. In some embodiments, the block copolymer can have a hardness of from about Shore 20A to about Shore 75D (e.g., from about Shore 40A to about Shore 90A). In certain embodiments, a copolymer with a desired degree of hardness may be formed by varying the proportions of the A and B blocks in the copolymer, with a lower relative proportion of B blocks resulting in a copolymer of lower hardness, and a higher relative proportion of B blocks resulting in a copolymer of higher hardness. As a specific example, high molecular weight (i.e., greater than 100,000 Daltons) polyisobutylene is a relatively soft and gummy material with a Shore hardness of approximately 10A. By comparison, polystyrene is much harder, typically having a Shore hardness on the order of 100D. As a result, when blocks of polyisobutylene and styrene are combined, the resulting copolymer can have a range of hardnesses from as soft as Shore 10A to as hard as Shore 100D, depending upon the relative amounts of polystyrene and polyisobutylene in the copolymer. In some embodiments, from about two mol percent to about 25 mol percent (e.g., from about five mol percent to about 20 mol percent) of polystyrene can be used to form a block copolymer with a hardness of from about Shore 30A to about Shore 90A (e.g., from about Shore 35A to about Shore 70A).

Polydispersity (the ratio of weight average molecular weight to number average molecular weight) gives an indication of the molecular weight distribution of a polymer, with values significantly greater than four indicating a broad molecular weight distribution. When all molecules within a sample are the same size, the polydispersity has a value of one. Typically, the polymers used in the particles described herein can have a relatively tight molecular weight distribution, with a polydispersity of from about 1.1 to about 1.7.

In some embodiments, one or more of the above-described polymers can have a relatively high tensile strength. For example, triblock copolymers of polystyrene-polyisobutylene-polystyrene can have a tensile strength of at least about 2,000 psi (e.g., from about 2,000 psi to about 4,000 psi).

In certain embodiments, one or more of the above-described polymers can be relatively resistant to cracking and/or other forms of degradation under in vivo conditions. Additionally or alternatively, one or more of the above-described polymers can exhibit excellent biocompatibility, including vascular compatibility. For example, the polymers can provoke minimal adverse tissue reactions, resulting in reduced polymorphonuclear leukocyte and reduced macrophage activity. In some embodiments, one or more of the above-described polymers can generally be hemocompatible, and can thereby minimize thrombotic occlusion of, for example, small vessels.

The above-described polymers can be made using any appropriate method known in the art. In some embodiments, the block copolymers, for example, can be made by a carbocationic polymerization process that includes an initial polymerization of a monomer or mixtures of monomers to form the A blocks, followed by the subsequent addition of a monomer or a mixture of monomers capable of forming the B blocks. Such polymerization reactions are described, for example, in Kennedy et al., U.S. Pat. No. 4,276,394; Kennedy, U.S. Pat. No. 4,316,973; Kennedy, 4,342,849; Kennedy et al., U.S. Pat. No. 4,910,321; Kennedy et al., U.S. Pat. No. 4,929,683; Kennedy et al., U.S. Pat. No. 4,946,899; Kennedy et al., U.S. Pat. No. 5,066,730; Kennedy et al., U.S. Pat. No. 5,122,572; and Kennedy et al., U.S. Pat. No. Re. 34,640. Each of these patents is incorporated herein by reference.

The techniques disclosed in these patents generally involve an “initiator”, which can be used to create X-(AB), structures, where X is the initiator, and n can be 1, 2, 3 or more. The initiator can be monofunctional or multifunctional. As noted above, the resulting molecules are referred to as diblock copolymers where n is 1, triblock copolymers (disregarding the presence of the initiator) where n is 2, and star-shaped block copolymers where n is 3 or more.

In general, the polymerization reaction can be conducted under conditions that minimize or avoid chain transfer and termination of the growing polymer chains. Steps can be taken to keep active hydrogen atoms (water, alcohol and the like) to a minimum. The temperature for the polymerization is usually from about −10° C. to about −90° C. (e.g., from about −60° C. to about −80° C.), although lower temperatures can be used.

Typically, one or more A blocks (e.g., polyisobutylene blocks) can be formed in a first step, followed by the addition of B blocks (e.g., polystyrene blocks) at the ends of the A blocks. More particularly, the first polymerization step is generally carried out in an appropriate solvent system, such as a mixture of polar and non-polar solvents (e.g., methyl chloride and hexanes). The reaction bath can contain the aforementioned solvent system, olefin monomer (e.g., isobutylene), an initiator (e.g., a tert-ester, tert-ether, tert-hydroxyl or tert-halogen containing compound, a cumyl ester of a hydrocarbon acid, an alkyl cumyl ether, a cumyl halide, a cumyl hydroxyl compound, or a hindered version of the above), and a coinitiator (e.g., a Lewis acid, such as boron trichloride or titanium tetrachloride). In some embodiments, electron pair donors (e.g., dimethyl acetamide, dimethyl sulfoxide, dimethyl phthalate) can be added to the solvent system. Additionally, proton-scavengers that scavenge water, such as 2,6-di-tert-butylpyridine, 4-methyl-2,6-di-tert-butylpyridine, 1,8-bis(dimethylamino)-naphthalene, or diisopropylethyl amine can be added.

The reaction is commenced by removing the tert-ester, tert-ether, tert-hydroxyl or tert-halogen (herein called the “tert-leaving groups”) from the initiator by reacting the initiator with the Lewis acid. In place of the tert-leaving groups is a quasi-stable or “living” cation which is stabilized by the surrounding tertiary carbons, as well as the polar solvent system and electron pair donors. After obtaining the cation, the A block monomer (e.g., isobutylene) is introduced, and cationically propagates or polymerizes from each cation on the initiator. When the A block is polymerized, the propagated cations remain on the ends of the A blocks. The B block monomer (e.g., styrene) is then introduced, and polymerizes and propagates from the ends of the A block. Once the B blocks are polymerized, the reaction is terminated by adding a termination molecule such as methanol, water and the like.

Product molecular weights are generally determined by reaction time, reaction temperature, the nature and concentration of the reactants, and so forth. Consequently, different reaction conditions may produce different products. In general, synthesis of the desired reaction product is achieved by an iterative process in which the course of the reaction is monitored by the examination of samples taken periodically during the reaction—a technique widely employed in the art. To achieve the desired product, an additional reaction may be required in which reaction time and temperature, reactant concentration, and so forth are changed.

Additional details regarding cationic processes for making copolymers are found, for example, in Kennedy et al., U.S. Pat. No. 4,276,394; Kennedy, U.S. Pat. No. 4,316,973; Kennedy, 4,342,849; Kennedy et al., U.S. Pat. No. 4,910,321; Kennedy et al., U.S. Pat. No. 4,929,683; Kennedy et al., U.S. Pat. No. 4,946,899; Kennedy et al., U.S. Pat. No. 5,066,730; Kennedy et al., U.S. Pat. No. 5,122,572; and Kennedy et al., U.S. Pat. No. Re. 34,640, incorporated supra.

The polymers may be recovered from a reaction mixture by any of the usual techniques including evaporation of solvent, precipitation with a non-solvent such as an alcohol or alcohol/acetone mixture, followed by drying, and so forth. In addition, purification of the polymers can be performed by sequential extraction in aqueous media, both with and without the presence of various alcohols, ethers and ketones.

In some embodiments, the particles described herein can be formed of a polymer that includes one or more functional groups. The functional groups can be negatively charged or positively charged, and/or can be ionically bonded to the polymer. In some embodiments, the functional groups can enhance the biocompatibility of the polymer. Alternatively or additionally, the functional groups can enhance the clot-forming capabilities of the polymer. Examples of functional groups include phosphate groups, carboxylate groups, sulfonate groups, sulfate groups, phosphonate groups, and phenolate groups. For example, a polymer can be a sulfonated styrenic polymer, such as sulfonated SIBS. Sulfonation of styrene containing polymers is disclosed, for example, in Ehrenberg, et al., U.S. Pat. No. 5,468,574; Vachon et al., U.S. Pat. No. 6,306,419; and Berlowitz-Tarrant, et al., U.S. Pat. No. 5,840,387, all of which are incorporated herein by reference. Examples of other functionalized polymers include phosphated SIBS and carboxylated SIBS. In certain embodiments, a polymer can include more than one different type of functional group. For example, a polymer can include both a sulfonate group and a phosphate group. In some embodiments, a polymer that includes a functional group can be reacted with a cross-linking and/or gelling agent during particle formation. For example, a particle that includes a sulfonates group, such as sulfonated SIBS, may be reacted with a cross-linking and/or gelling agent such as calcium chloride. Functionalized polymers and cross-linking and/or gelling agents are described, for example, in Richard et al., U.S. patent application Ser. No. 10/927,868, filed on Aug. 27, 2004, and entitled “Embolization”, which is incorporated herein by reference.

In certain embodiments, the polymer can include a highly water insoluble, high molecular weight polymer. An example of such a polymer is a high molecular weight PVA that has been acetalized. The polymer can include substantially pure intrachain 1,3-acetalized PVA, and can be substantially free of animal derived residue such as collagen. In some embodiments, the polymer can include a minor amount (e.g., about 2.5 weight percent or less, about one weight percent or less, about 0.2 weight percent or less) of a gelling material (e.g., a polysaccharide, such as alginate). In certain embodiments, the polymer can include a bioabsorbable (e.g., resorbable) polymer (e.g., alginate, gelatin, albumin, resorbable polyvinyl alcohol, albumin, dextran, starch, ethyl cellulose, polyglycolic acid, polylactic acid, polylactic acid/polyglycolic acid copolymers, poly(lactic-co-glycolic) acid). The polymer can include, for example, polyvinyl alcohol, alginate, or both polyvinyl alcohol and alginate. The polymer can further include a wax.

As described above, the particle 100 can be used to deliver one or more therapeutic agents (e.g., a combination of therapeutic agents) to a target site. Therapeutic agents include genetic therapeutic agents, non-genetic therapeutic agents, and cells, and can be negatively charged, positively charged, amphoteric, or neutral. Therapeutic agents can be, for example, materials that are biologically active to treat physiological conditions; pharmaceutically active compounds; proteins; gene therapies; nucleic acids with and without carrier vectors (e.g., recombinant nucleic acids, DNA (e.g., naked DNA), cDNA, RNA, genomic DNA, cDNA or RNA in a non-infectious vector or in a viral vector which may have attached peptide targeting sequences, antisense nucleic acids (RNA, DNA)); oligonucleotides; gene/vector systems (e.g., anything that allows for the uptake and expression of nucleic acids); DNA chimeras (e.g., DNA chimeras which include gene sequences and encoding for ferry proteins such as membrane translocating sequences (“MTS”) and herpes simplex virus-1 (“VP22”)); compacting agents (e.g., DNA compacting agents); viruses; polymers; hyaluronic acid; proteins (e.g., enzymes such as ribozymes, asparaginase); immunologic species; nonsteroidal anti-inflammatory medications; oral contraceptives; progestins; gonadotrophin-releasing hormone agonists; chemotherapeutic agents; and radioactive species (e.g., radioisotopes, radioactive molecules). Examples of radioactive species include yttrium (⁹⁰Y), holmium (¹⁶⁶Ho), phosphorus (³²P), (¹⁷⁷Lu), actinium (²²⁵Ac), praseodymium, astatine (²¹¹At), rhenium (¹⁸⁶Re), bismuth (²¹²Bi or ²¹³Bi),), samarium (¹⁵³Sm), iridium (¹⁹²Ir), rhodium (¹⁰⁵Rh), iodine (^{131,} or ¹²⁵I), indium (¹¹¹In), technetium (⁹⁹Tc), phosphorus (³²P), sulfur (³⁵S), carbon (¹⁴C), tritium (³H), chromium (⁵¹Cr), chlorine (³⁶Cl), cobalt (⁵⁷Co or ⁵³Co), iron (⁵⁹Fe), selenium (⁷⁵Se), and/or gallium (⁶⁷ Ga). In some embodiments, yttrium (⁹⁰Y), lutetium (¹⁷⁷ Lu), actinium (²²⁵ Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), and/or rhodium (¹⁰⁵Rh) can be used as therapeutic agents. In certain embodiments, yttrium (⁹⁰Y), lutetium (¹⁷⁷Lu), actinium (²²⁵Ac), praseodymium, astatine (²¹¹At), rhenium (¹⁸⁶Re), bismuth (²¹²Bi or ²¹³Bi), holmium (¹⁶⁶Ho), samarium (¹⁵³Sm), iridium (¹⁹²Ir), rhodium (¹⁰⁵Rh), iodine (¹³¹I or ¹²⁵I), indium (¹¹¹In), technetium (⁹⁹Tc), phosphorus (³²P), carbon (¹⁴C), and/or tritium (³H) can be used as a radioactive label (e.g., for use in diagnostics). In some embodiments, a radioactive species can be a radioactive molecule that includes antibodies containing one or more radioisotopes, for example, a radiolabeled antibody. Radioisotopes that can be bound to antibodies include, for example, iodine (¹³¹I or ¹²⁵I), yttrium (⁹⁰Y), lutetium (¹⁷⁷ Lu), actinium (²²⁵ Ac), praseodymium, astatine (²¹¹At), rhenium (¹⁸⁶Re), bismuth (²¹²Bi or ²¹³Bi), indium (¹¹¹In), technetium (⁹⁹Tc), phosphorus (³²P), rhodium (¹⁰⁵Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), and/or gallium (⁶⁷Ga). Examples of antibodies include monoclonal and polyclonal antibodies including RS7, Mov18, MN-14 IgG, CC49, COL-1, mAB A33, NP-4 F(ab′)2 anti-CEA, anti-PSMA, ChL6, m-170, or antibodies to CD20, CD74 or CD52 antigens. Examples of radioisotope/antibody pairs include m-170 MAB with ⁹⁰Y. Examples of commercially available radioisotope/antibody pairs include Zevalin™ (IDEC pharmaceuticals, San Diego, Calif.) and Bexxar™ (Corixa corporation, Seattle, Wash.). Further examples of radioisotope/antibody pairs can be found in J. Nucl. Med. 2003, Apr: 44(4): 632-40.

Non-limiting examples of therapeutic agents include anti-thrombogenic agents; thrombogenic agents; agents that promote clotting; agents that inhibit clotting; antioxidants; angiogenic and anti-angiogenic agents and factors; anti-proliferative agents (e.g., agents capable of blocking smooth muscle cell proliferation, such as rapamycin); calcium entry blockers (e.g., verapamil, diltiazem, nifedipine); targeting factors (e.g., polysaccharides, carbohydrates); agents that can stick to the vasculature (e.g., charged moieties, such as gelatin, chitosan, and collagen); and survival genes which protect against cell death (e.g., anti-apoptotic Bcl-2 family factors and Akt kinase).

Examples of non-genetic therapeutic agents include: anti-thrombotic agents such as heparin, heparin derivatives, urokinase, and PPack (dextrophenylalanine proline arginine chloromethylketone); anti-inflammatory agents such as dexamethasone, prednisolone, corticosterone, budesonide, estrogen, acetyl salicylic acid, sulfasalazine and mesalamine; antineoplastic/antiproliferative/anti-mitotic agents such as paclitaxel, 5-fluorouracil, cisplatin, methotrexate, doxorubicin, vinblastine, vincristine, epothilones, endostatin, angiostatin, angiopeptin, monoclonal antibodies capable of blocking smooth muscle cell proliferation, and thymidine kinase inhibitors; anesthetic agents such as lidocaine, bupivacaine and ropivacaine; anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet factors or peptides; vascular cell growth promoters such as growth factors, transcriptional activators, and translational promoters; vascular cell growth inhibitors such as growth factor inhibitors (e.g., PDGF inhibitor-Trapidil), growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; protein kinase and tyrosine kinase inhibitors (e.g., tyrphostins, genistein, quinoxalines); prostacyclin analogs; cholesterol-lowering agents; angiopoietins; antimicrobial agents such as triclosan, cephalosporins, aminoglycosides and nitrofurantoin; cytotoxic agents, cytostatic agents and cell proliferation affectors; vasodilating agents; and agents that interfere with endogenous vasoactive mechanisms.

Examples of genetic therapeutic agents include: anti-sense DNA and RNA; DNA coding for anti-sense RNA, tRNA or rRNA to replace defective or deficient endogenous molecules, angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor a, hepatocyte growth factor, and insulin like growth factor, cell cycle inhibitors including CD inhibitors, thymidine kinase (“TK”) and other agents useful for interfering with cell proliferation, and the family of bone morphogenic proteins (“BMP's”), including BMP2, BMP3, BMP4, BMP5, BMP6 (Vgr1), BMP7 (OP1), BMP8, BMP9, BMP10, BM11, BMP12, BMP13, BMP14, BMP15, and BMP16. Currently preferred BMP's are any of BMP2, BMP3, BMP4, BMP5, BMP6 and BMP7. These dimeric proteins can be provided as homodimers, heterodimers, or combinations thereof, alone or together with other molecules. Alternatively or additionally, molecules capable of inducing an upstream or downstream effect of a BMP can be provided. Such molecules include any of the “hedgehog” proteins, or the DNA's encoding them.

Vectors of interest for delivery of genetic therapeutic agents include: plasmids; viral vectors such as adenovirus (AV), adenoassociated virus (AAV) and lentivirus; and non-viral vectors such as lipids, liposomes, and cationic lipids.

Cells include cells of human origin (autologous or allogeneic), including stem cells, or from an animal source (xenogeneic), which can be genetically engineered if desired to deliver proteins of interest.

Several of the above and numerous additional therapeutic agents are disclosed in Kunz et al., U.S. Pat. No. 5,733,925, which is incorporated herein by reference. Therapeutic agents disclosed in this patent include the following:

“Cytostatic agents” (i.e., agents that prevent or delay cell division in proliferating cells, for example, by inhibiting replication of DNA or by inhibiting spindle fiber formation). Representative examples of cytostatic agents include modified toxins, methotrexate, adriamycin, radionuclides (e.g., such as disclosed in Fritzberg et al., U.S. Pat. No. 4,897,255), protein kinase inhibitors, including staurosporin, a protein kinase C inhibitor of the following formula:

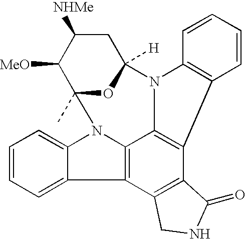

as well as diindoloalkaloids having one of the following general structures:

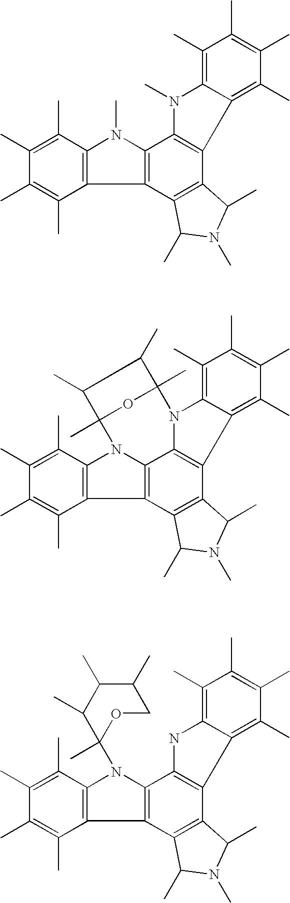

as well as stimulators of the production or activation of TGF-beta, including Tamoxifen and derivatives of functional equivalents (e.g., plasmin, heparin, compounds capable of reducing the level or inactivating the lipoprotein Lp(a) or the glycoprotein apolipoprotein(a)) thereof, TGF-beta or functional equivalents, derivatives or analogs thereof, suramin, nitric oxide releasing compounds (e.g., nitroglycerin) or analogs or functional equivalents thereof, paclitaxel or analogs thereof (e.g., taxotere), inhibitors of specific enzymes (such as the nuclear enzyme DNA topoisomerase II and DNA polymerase, RNA polymerase, adenyl guanyl cyclase), superoxide dismutase inhibitors, terminal deoxynucleotidyl-transferase, reverse transcriptase, antisense oligonucleotides that suppress smooth muscle cell proliferation and the like. Other examples of “cytostatic agents” include peptidic or mimetic inhibitors (i.e., antagonists, agonists, or competitive or non-competitive inhibitors) of cellular factors that may (e.g., in the presence of extracellular matrix) trigger proliferation of smooth muscle cells or pericytes: e.g., cytokines (e.g., interleukins such as IL-1), growth factors (e.g., PDGF, TGF-alpha or -beta, tumor necrosis factor, smooth muscle- and endothelial-derived growth factors, i.e., endothelin, FGF), homing receptors (e.g., for platelets or leukocytes), and extracellular matrix receptors (e.g., integrins). Representative examples of useful therapeutic agents in this category of cytostatic agents addressing smooth muscle proliferation include: subfragments of heparin, triazolopyrimidine (trapidil; a PDGF antagonist), lovastatin, and prostaglandins E1 or I2.

Agents that inhibit the intracellular increase in cell volume (i.e., the tissue volume occupied by a cell), such as cytoskeletal inhibitors or metabolic inhibitors. Representative examples of cytoskeletal inhibitors include colchicine, vinblastin, cytochalasins, paclitaxel and the like, which act on microtubule and microfilament networks within a cell. Representative examples of metabolic inhibitors include staurosporin, trichothecenes, and modified diphtheria and ricin toxins, Pseudomonas exotoxin and the like. Trichothecenes include simple trichothecenes (i. e., those that have only a central sesquiterpenoid structure) and macrocyclic trichothecenes (i. e., those that have an additional macrocyclic ring), e.g., a verrucarins or roridins, including Verrucarin A, Verrucarin B, Verrucarin J (Satratoxin C), Roridin A, Roridin C, Roridin D, Roridin E (Satratoxin D), Roridin H.

Agents acting as an inhibitor that blocks cellular protein synthesis and/or secretion or organization of extracellular matrix (i.e., an “anti-matrix agent”). Representative examples of “anti-matrix agents” include inhibitors (i.e., agonists and antagonists and competitive and non-competitive inhibitors) of matrix synthesis, secretion and assembly, organizational cross-linking (e.g., transglutaminases cross-linking collagen), and matrix remodeling (e.g., following wound healing). A representative example of a useful therapeutic agent in this category of anti-matrix agents is colchicine, an inhibitor of secretion of extracellular matrix. Another example is tamoxifen for which evidence exists regarding its capability to organize and/or stabilize as well as diminish smooth muscle cell proliferation following angioplasty. The organization or stabilization may stem from the blockage of vascular smooth muscle cell maturation in to a pathologically proliferating form.

Agents that are cytotoxic to cells, particularly cancer cells. Preferred agents are Roridin A, Pseudomonas exotoxin and the like or analogs or functional equivalents thereof. A plethora of such therapeutic agents, including radioisotopes and the like, have been identified and are known in the art. In addition, protocols for the identification of cytotoxic moieties are known and employed routinely in the art.

A number of the above therapeutic agents and several others have also been identified as candidates for vascular treatment regimens, for example, as agents targeting restenosis. Such agents include one or more of the following: calcium-channel blockers, including benzothiazapines (e.g., diltiazem, clentiazem); dihydropyridines (e.g., nifedipine, amlodipine, nicardapine); phenylalkylamines (e.g., verapamil); serotonin pathway modulators, including 5-HT antagonists (e.g., ketanserin, naftidrofuryl) and 5-HT uptake inhibitors (e.g., fluoxetine); cyclic nucleotide pathway agents, including phosphodiesterase inhibitors (e.g., cilostazole, dipyridamole), adenylate/guanylate cyclase stimulants (e.g., forskolin), and adenosine analogs; catecholamine modulators, including α-antagonists (e.g., prazosin, bunazosine), β-antagonists (e.g., propranolol), and α/β-antagonists (e.g., labetalol, carvedilol); endothelin receptor antagonists; nitric oxide donors/releasing molecules, including organic nitrates/nitrites (e.g., nitroglycerin, isosorbide dinitrate, amyl nitrite), inorganic nitroso compounds (e.g., sodium nitroprusside), sydnonimines (e.g., molsidomine, linsidomine), nonoates (e.g., diazenium diolates, NO adducts of alkanediamines), S-nitroso compounds, including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), C-nitroso-, O-nitroso- and N-nitroso-compounds, and L-arginine; ACE inhibitors (e.g., cilazapril, fosinopril, enalapril); ATII-receptor antagonists (e.g., saralasin, losartin); platelet adhesion inhibitors (e.g., albumin, polyethylene oxide); platelet aggregation inhibitors, including aspirin and thienopyridine (ticlopidine, clopidogrel) and GP Iib/IIIa inhibitors (e.g., abciximab, epitifibatide, tirofiban, intergrilin); coagulation pathway modulators, including heparinoids (e.g., heparin, low molecular weight heparin, dextran sulfate, β-cyclodextrin tetradecasulfate), thrombin inhibitors (e.g., hirudin, hirulog, PPACK (D-phe-L-propyl-L-arg-chloromethylketone), argatroban), Fxa inhibitors (e.g., antistatin, TAP (tick anticoagulant peptide)), vitamin K inhibitors (e.g., warfarin), and activated protein C; cyclooxygenase pathway inhibitors (e.g., aspirin, ibuprofen, flurbiprofen, indomethacin, sulfinpyrazone); natural and synthetic corticosteroids (e.g., dexamethasone, prednisolone, methprednisolone, hydrocortisone); lipoxygenase pathway inhibitors (e.g., nordihydroguairetic acid, caffeic acid; leukotriene receptor antagonists; antagonists of E- and P-selectins; inhibitors of VCAM-1 and ICAM-1 interactions; prostaglandins and analogs thereof, including prostaglandins such as PGE 1 and PGI2; prostacyclins and prostacyclin analogs (e.g., ciprostene, epoprostenol, carbacyclin, iloprost, beraprost); macrophage activation preventers (e.g., bisphosphonates); HMG-CoA reductase inhibitors (e.g., lovastatin, pravastatin, fluvastatin, simvastatin, cerivastatin); fish oils and omega-3-fatty acids; free-radical scavengers/antioxidants (e.g., probucol, vitamins C and E, ebselen, retinoic acid (e.g., trans-retinoic acid), SOD mimics); agents affecting various growth factors including FGF pathway agents (e.g., bFGF antibodies, chimeric fusion proteins), PDGF receptor antagonists (e.g., trapidil), IGF pathway agents (e.g., somatostatin analogs such as angiopeptin and ocreotide), TGF-β pathway agents such as polyanionic agents (heparin, fucoidin), decorin, and TGF-β antibodies, EGF pathway agents (e.g., EGF antibodies, receptor antagonists, chimeric fusion proteins), TNF-α pathway agents (e.g., thalidomide and analogs thereof), thromboxane A2 (TXA2) pathway modulators (e.g., sulotroban, vapiprost, dazoxiben, ridogrel), protein tyrosine kinase inhibitors (e.g., tyrphostin, genistein, and quinoxaline derivatives); MMP pathway inhibitors (e.g., marimastat, ilomastat, metastat), and cell motility inhibitors (e.g., cytochalasin B); antiproliferative/antineoplastic agents including antimetabolites such as purine analogs (e.g., 6-mercaptopurine), pyrimidine analogs (e.g., cytarabine and 5-fluorouracil) and methotrexate, nitrogen mustards, alkyl sulfonates, ethylenimines, antibiotics (e.g., daunorubicin, doxorubicin, daunomycin, bleomycin, mitomycin, penicillins, cephalosporins, ciprofalxin, vancomycins, aminoglycosides, quinolones, polymyxins, erythromycins, tertacyclines, chloramphenicols, clindamycins, linomycins, sulfonamides, and their homologs, analogs, fragments, derivatives, and pharmaceutical salts), nitrosoureas (e.g., carmustine, lomustine) and cisplatin, agents affecting microtubule dynamics (e.g., vinblastine, vincristine, colchicine, paclitaxel, epothilone), caspase activators, proteasome inhibitors, angiogenesis inhibitors (e.g., endostatin, angiostatin and squalamine), and rapamycin, cerivastatin, flavopiridol and suramin; matrix deposition/organization pathway inhibitors (e.g., halofuginone or other quinazolinone derivatives, tranilast); endothelialization facilitators (e.g., VEGF and RGD peptide); and blood rheology modulators (e.g., pentoxifylline).

Other examples of therapeutic agents include anti-tumor agents, such as docetaxel, alkylating agents (e.g., mechlorethamine, chlorambucil, cyclophosphamide, melphalan, ifosfamide), plant alkaloids (e.g., etoposide), inorganic ions (e.g., cisplatin), biological response modifiers (e.g., interferon), and hormones (e.g., tamoxifen, flutamide), as well as their homologs, analogs, fragments, derivatives, and pharmaceutical salts.

Additional examples of therapeutic agents include organic-soluble therapeutic agents, such as mithramycin, cyclosporine, and plicamycin. Further examples of therapeutic agents include pharmaceutically active compounds, anti-sense genes, viral, liposomes and cationic polymers (e.g., selected based on the application), biologically active solutes (e.g., heparin), prostaglandins, prostcyclins, L-arginine, nitric oxide (NO) donors (e.g., lisidomine, molsidomine, NO-protein adducts, NO-polysaccharide adducts, polymeric or oligomeric NO adducts or chemical complexes), enoxaparin, Warafin sodium, dicumarol, interferons, interleukins, chymase inhibitors (e.g., Tranilast), ACE inhibitors (e.g., Enalapril), serotonin antagonists, 5-HT uptake inhibitors, and beta blockers, and other antitumor and/or chemotherapy drugs, such as BiCNU, busulfan, carboplatinum, cisplatinum, cytoxan, DTIC, fludarabine, mitoxantrone, velban, VP-16, herceptin, leustatin, navelbine, rituxan, and taxotere.

In some embodiments, a therapeutic agent can be hydrophilic. An example of a hydrophilic therapeutic agent is doxorubicin hydrochloride. In certain embodiments, a therapeutic agent can be hydrophobic. Examples of hydrophobic therapeutic agents include paclitaxel, cisplatin, tamoxifen, and doxorubicin base. In some embodiments, a therapeutic agent can be lipophilic. Examples of lipophilic therapeutic agents include taxane derivatives (e.g., paclitaxel) and steroidal materials (e.g., dexamethasone).

Therapeutic agents are described, for example, in DiMatteo et al., U.S. Patent Application Publication No. US 2004/0076582 A1, published on Apr. 22, 2004, and entitled “Agent Delivery Particle”; Schwarz et al., U.S. Pat. No. 6,368,658; Buiser et al., U.S. patent application Ser. No. 11/311,617, filed on Dec. 19, 2005, and entitled “Coils”; and Song, U.S. patent application Ser. No. 11/355,301, filed on Feb. 15, 2006, and entitled “Block Copolymer Particles”, all of which are incorporated herein by reference. In certain embodiments, in addition to or as an alternative to including therapeutic agents, the particle 100 can include one or more radiopaque materials, materials that are visible by magnetic resonance imaging (MRI-visible materials), ferromagnetic materials, and/or contrast agents (e.g., ultrasound contrast agents). Radiopaque materials, MRI-visible materials, ferromagnetic materials, and contrast agents are described, for example, in Rioux et al., U.S. Patent Application Publication No. US 2004/0101564 A1, published on May 27, 2004, and entitled “Embolization”, which is incorporated herein by reference.

Particles can be formed by any of a number of different methods.

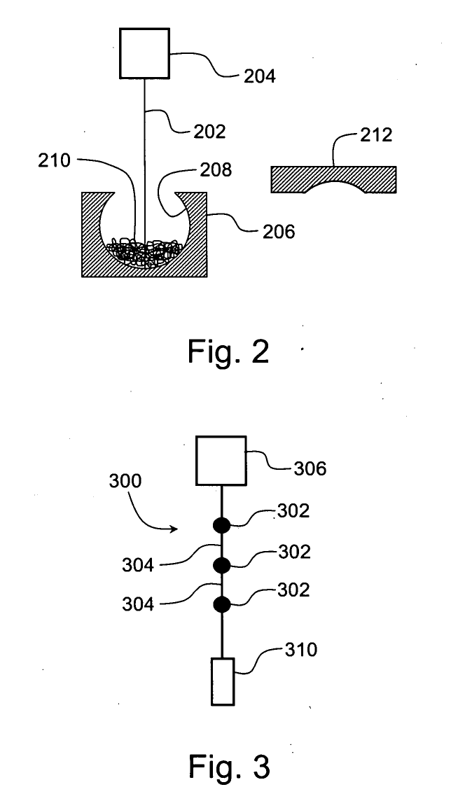

One method of making a particle as described above with respect to FIG. 1 is illustrated in FIG. 2. In this method an electrostatically charged polymer fiber strand 202 is extruded from an extruder nozzle 204 into a mold 206 with an electrostatically charged interior surface 208. The electrostatically charged polymer fiber strand 202 in this method is electrostatically attracted to the interior surface 208 of the mold 206, i.e., the electrostatically charged polymer fiber strand 202 and the electrostatically charged interior surface 208 are oppositely charged. The surface portions of the mold 206 other than the electrostatically charged interior surface 208 can be insulated. When the surfaces of the mold 206 other than the electrostatically charged interior surface 208 are insulated, the electrostatically charged polymer fiber 202 is only attracted to the interior portion of the mold. As the mold is filled with the polymer fiber strand 202 a particle with the properties described above is formed. An incomplete portion of a particle 210 is shown in FIG. 2.

The mold 206 can be designed to produce particles with different sizes or shapes (e.g., spherical or elliptical). After a particle is formed in the mold 206, the mold 206 can be heated thereby heating the particle. Applying enough heat to a mold to cause the polymer to soften, but not melt completely, can be used to create intra-strand and/or inter-strand bonds. Particles can also be heated enough that the overall size of the particle is reduced. The mold 206 can also be designed with a lid portion 212 that is positionable on the mold 206 after the mold 206 is filled with the polymer fiber strand 202 to complete the shape of the upper portion of the particle.

In general, a mold 206 can have a largest dimension of 5,000 microns or less (e.g., from two microns to 5,000 microns; from 10 microns to 5,000 microns; from 40 microns to 2,000 microns; from 100 microns to 700 microns; from 500 microns to 700 microns; from 100 microns to 500 microns; from 100 microns to 300 microns; from 300 microns to 500 microns; from 500 microns to 1,200 microns; from 500 microns to 700 microns; from 700 microns to 900 microns; from 900 microns to 1,200 microns; from 1,000 microns to 1,200 microns). In some embodiments, the largest dimension of a mold 206 is 5,000 microns or less (e.g., 4,500 microns or less, 4,000 microns or less, 3,500 microns or less, 3,000 microns or less, 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the largest dimension of a mold 206 is less than 100 microns (e.g., less than 50 microns).

This method can also be used to form particles that include a core portion. To make a particle with a core, a pre-formed core is placed in the mold 206 either before or during introduction of the polymer fiber strand 202. Once the core is place in the mold 206, the polymer fiber strand 202 falls about the core thereby enveloping the core within the particle as the particle is formed.

This method has been described thus far using a single polymer fiber strand 202, however, extruder nozzles 204 that generate multiple fiber strands are contemplated. Further the use of multiple extruder nozzles fed by multiple extruders extruding different types of polymers is contemplated.

A further embodiment of this method can include the additional step of combining the particles with pharmaceutically acceptable media, therapeutic agents, radiopaque materials, materials that are visible by magnetic resonance imaging (MRI-visible materials), ferromagnetic materials, and/or contrast agents (e.g., ultrasound contrast agents).

An additional embodiment of this method can include connecting the particle that is formed to a second particle by forming a link between the particles. Particle chains and methods of making particle chains are described, for example, in Buiser et al., U.S. Patent Application Publication No. US 2005/0238870 A1, published on Oct. 27, 2005, and entitled “Embolization,” which is incorporated herein by reference.

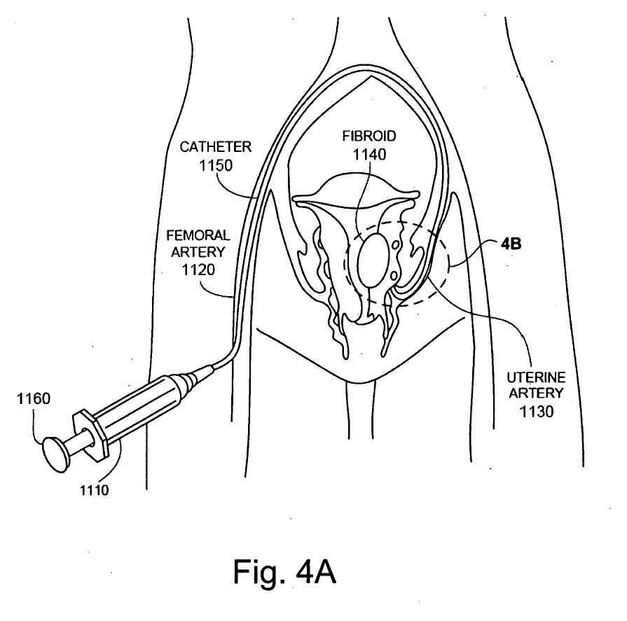

A method for making a particle chain is also disclosed herein. In this method, a particle chain is formed by sequentially extruding particle portions and linkage portions from an extruder nozzle. As shown in FIG. 3, a particle chain 300 including particle portions 302 and linkage portions 304 is formed by extruding a particle portion 302 then extruding a linkage portion 304 from an extruder nozzle 306 without disconnecting the particle portions 302 from the linkage portions 304. Particle portions 302 and linkage portions 304 are formed on the same particle chain 300 by adjusting the flow rate of the polymer being extruded from the extruder nozzle 306. In general, when the relative flow rate is decreased a particle portion 302 is formed and when the relative flow rate is increased (or brought back to the starting flow rate) a linkage portion 304 is formed. The size of the particle portion 302 and the size and aspect ratio of the linkage portion 304 can be controlled by controlling the flow rate.

Several methods for altering the flow rate of a polymer being extruded from an extruder nozzle 306 exist. For example, the force being applied to the polymer by the extruder can be altered, the inside largest dimension of the nozzle 306 can be altered, or the speed at which the polymer is drawn from the nozzle 306 can be altered. The force being applied to the polymer by the extruder can be altered, for example, by varying the rate at which the extruder screw turns thereby altering the polymer pressure at the nozzle 306. The inside diameter of the nozzle 306 can be altered, for example, through mechanical reducing or increasing the diameter. The speed at which the polymer is drawn from the nozzle 306 can be altered, for example, by electrostatically charging the polymer and electrostatically charging a takeup portion 310, then varying the potential difference between the nozzle and the takeup 310. The takeup portion 310 can be, for example, an electrode. Additionally, two or more of these techniques for varying the flow rate can be combined to effect flow rate change.

The same polymers, additives (such as waxes and alginate), and therapeutic agents useful with the particle 100 described above are useful with these methods. The physical characteristics, i.e., largest dimension, length, aspect ratio, etc., of particle chains formed by this method are the same as those described above for particle chain 110.

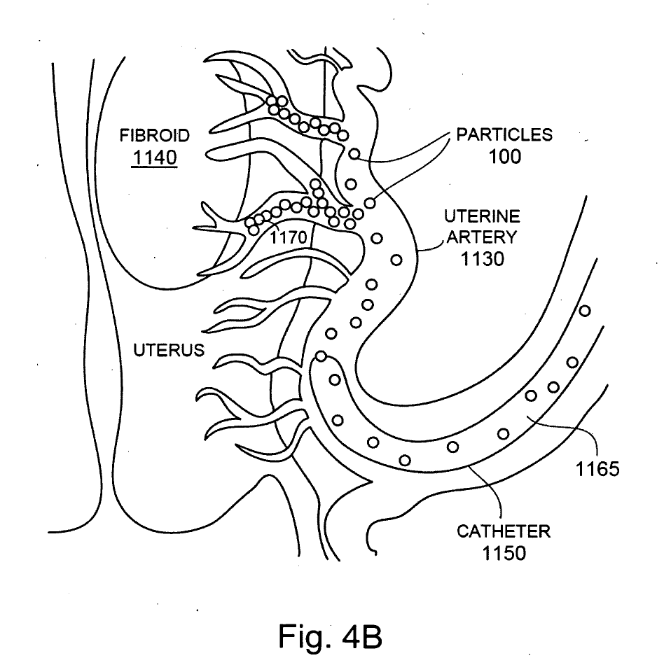

In some embodiments, in addition to or as an alternative to being used to deliver a therapeutic agent to a target site, the particle 100 or particle chain 300 can be used to embolize a target site (e.g., a lumen of a subject). For example, multiple particles can be combined with a carrier fluid (e.g., a pharmaceutically acceptable carrier, such as a saline solution, a contrast agent, or both) to form a composition, which can then be delivered to a site and used to embolize the site. FIGS. 4A and 4B illustrate the use of a composition including particles to embolize a lumen of a subject. As shown, a composition, including particles 100 or particle chain 300 and a carrier fluid, is injected into a vessel through an instrument such as a catheter 1150. Catheter 1150 is connected to a syringe barrel 1110 with a plunger 1160. Catheter 1150 is inserted, for example, into a femoral artery 1120 of a subject. Catheter 1150 delivers the composition to, for example, occlude a uterine artery 1130 leading to a fibroid 1140. Fibroid 1140 is located in the uterus of a female subject. The composition is initially loaded into syringe 1110. Plunger 1160 of syringe 1110 is then compressed to deliver the composition through catheter 1150 into a lumen 1165 of uterine artery 1130.

FIG. 4B, which is an enlarged view of section 4B of FIG. 4A, shows a uterine artery 1130 that is subdivided into smaller uterine vessels 1170 (e.g., having a largest dimension of about two millimeters or less) which feed fibroid 1140. The particles 100 or particle chain 110 in the composition partially or totally fill the lumen of uterine artery 1130, either partially or completely occluding the lumen of the uterine artery 1130 that feeds uterine fibroid 1140.

Compositions that include particles such as particles 100 or particle chain 110 can be delivered to various sites in the body, including, for example, sites having cancerous lesions, such as the breast, prostate, lung, thyroid, or ovaries. The compositions can be used in, for example, neural, pulmonary, and/or AAA (abdominal aortic aneurysm) applications. The compositions can be used in the treatment of, for example, fibroids, tumors, internal bleeding, arteriovenous malformations (AVMs), and/or hypervascular tumors. The compositions can be used as, for example, fillers for aneurysm sacs, AAA sac (Type II endoleaks), endoleak sealants, arterial sealants, and/or puncture sealants, and/or can be used to provide occlusion of other lumens such as fallopian tubes. Fibroids can include uterine fibroids which grow within the uterine wall (intramural type), on the outside of the uterus (subserosal type), inside the uterine cavity (submucosal type), between the layers of broad ligament supporting the uterus (interligamentous type), attached to another organ (parasitic type), or on a mushroom-like stalk (pedunculated type). Internal bleeding includes gastrointestinal, urinary, renal and varicose bleeding. AVMs are for example, abnormal collections of blood vessels, e.g. in the brain, which shunt blood from a high pressure artery to a low pressure vein, resulting in hypoxia and malnutrition of those regions from which the blood is diverted. In some embodiments, a composition containing the particles can be used to prophylactically treat a condition.

The magnitude of a dose of a composition can vary based on the nature, location and severity of the condition to be treated, as well as the route of administration. A physician treating the condition, disease or disorder can determine an effective amount of composition. An effective amount of embolic composition refers to the amount sufficient to result in amelioration of symptoms and/or a prolongation of survival of the subject, or the amount sufficient to prophylactically treat a subject. The compositions can be administered as pharmaceutically acceptable compositions to a subject in any therapeutically acceptable dosage, including those administered to a subject intravenously, subcutaneously, percutaneously, intratrachealy, intramuscularly, intramucosaly, intracutaneously, intra-articularly, orally or parenterally.

A composition can include a mixture of particles or particle chains (e.g., particles or particle chains that include different types of block copolymers, particles that include different types of therapeutic agents), or can include particles that are all of the same type. In some embodiments, a composition can be prepared with a calibrated concentration of particles or particle chains for ease of delivery by a physician. A physician can select a composition of a particular concentration based on, for example, the type of procedure to be performed. In certain embodiments, a physician can use a composition with a relatively high concentration of particles or particle chains during one part of an embolization procedure, and a composition with a relatively low concentration of particles or particle chains during another part of the embolization procedure.

Suspensions of particles or particle chains in saline solution can be prepared to remain stable (e.g., to remain suspended in solution and not settle and/or float) over a desired period of time. A suspension of particles or particle chains can be stable, for example, for from about one minute to about 20 minutes (e.g. from about one minute to about 10 minutes, from about two minutes to about seven minutes, from about three minutes to about six minutes).

In some embodiments, particles or particle chains can be suspended in a physiological solution by matching the density of the solution to the density of the particles or particle chains. In certain embodiments, the particles or particle chains and/or the physiological solution can have a density of from about one gram per cubic centimeter to about 1.5 grams per cubic centimeter (e.g., from about 1.2 grams per cubic centimeter to about 1.4 grams per cubic centimeter, from about 1.2 grams per cubic centimeter to about 1.3 grams per cubic centimeter).

In some embodiments, the carrier fluid of a composition can include a surfactant. The surfactant can help the particles or particle chains to mix evenly in the carrier fluid and/or can decrease the likelihood of the occlusion of a delivery device (e.g., a catheter) by the particles. In certain embodiments, the surfactant can enhance delivery of the composition (e.g., by enhancing the wetting properties of the particles or particle chains and facilitating the passage of the particles through a delivery device). In some embodiments, the surfactant can decrease the occurrence of air entrapment by the particles or particle chains in a composition (e.g., by porous particles in a composition). Examples of liquid surfactants include Tween® 80 (available from Sigma-Aldrich) and Cremophor EL® (available from Sigma-Aldrich). An example of a powder surfactant is Pluronic® F127 NF (available from BASF). In certain embodiments, a composition can include from about 0.05 percent by weight to about one percent by weight (e.g., about 0.1 percent by weight, about 0.5 percent by weight) of a surfactant. A surfactant can be added to the carrier fluid prior to mixing with the particles or particle chains and/or can be added to the particles or particle chains prior to mixing with the carrier fluid.

In some embodiments, among the particles delivered to a subject (e.g., in a composition), the majority (e.g., 50 percent or more, 60 percent or more, 70 percent or more, 80 percent or more, 90 percent or more) of the particles can have a largest dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, among the particles delivered to a subject, the majority of the particles can have a largest dimension of less than 100 microns (e.g., less than 50 microns).

In certain embodiments, the particles delivered to a subject (e.g., in a composition) can have an arithmetic mean largest dimension of 5,000 microns or less (e.g., 4,500 microns or less; 4,000 microns or less; 3,500 microns or less; 3,000 microns or less; 2,500 microns or less; 2,000 microns or less; 1,500 microns or less; 1,200 microns or less; 1,150 microns or less; 1,100 microns or less; 1,050 microns or less; 1,000 microns or less; 900 microns or less; 700 microns or less; 500 microns or less; 400 microns or less; 300 microns or less; 100 microns or less; 50 microns or less; 10 microns or less; five microns or less) and/or one micron or more (e.g., five microns or more; 10 microns or more; 50 microns or more; 100 microns or more; 300 microns or more; 400 microns or more; 500 microns or more; 700 microns or more; 900 microns or more; 1,000 microns or more; 1,050 microns or more; 1,100 microns or more; 1,150 microns or more; 1,200 microns or more; 1,500 microns or more; 2,000 microns or more; 2,500 microns or more). In some embodiments, the particles delivered to a subject can have an arithmetic mean largest dimension of less than 100 microns (e.g., less than 50 microns).

Exemplary ranges for the arithmetic mean largest dimension of particles or particle portions of particle chains delivered to a subject include from about 100 microns to about 500 microns; from about 100 microns to about 300 microns; from about 300 microns to about 500 microns; from about 500 microns to about 700 microns; from about 700 microns to about 900 microns; from about 900 microns to about 1,200 microns; and from about 1,000 microns to about 1,200 microns. In general, the particles or particle portions of particle chains delivered to a subject (e.g., in a composition) can have an arithmetic mean largest dimension in approximately the middle of the range of the largest dimensions of the individual particles or particle portions of particle chains, and a variance of about 20 percent or less (e.g. about 15 percent or less, about 10 percent or less).

In some embodiments, the arithmetic mean largest dimension of the particles or particle portions of particle chains delivered to a subject (e.g., in a composition) can vary depending upon the particular condition to be treated. As an example, in embodiments in which the particles or particle chains are used to embolize a liver tumor, the particles or particle portions of particle chains delivered to the subject can have an arithmetic mean largest dimension of about 500 microns or less (e.g., from about 100 microns to about 300 microns; from about 300 microns to about 500 microns). As another example, in embodiments in which the particles or particle chains are used to embolize a uterine fibroid, the particles or particle portions of particle chains delivered to the subject can have an arithmetic mean largest dimension of about 1,200 microns or less (e.g., from about 500 microns to about 700 microns; from about 700 microns to about 900 microns; from about 900 microns to about 1,200 microns). As an additional example, in embodiments in which the particles or particle chains are used to treat a neural condition (e.g., a brain tumor) and/or head trauma (e.g., bleeding in the head), the particles or particle portions of particle chains delivered to the subject can have an arithmetic mean largest dimension of less than about 100 microns (e.g., less than about 50 microns). As a further example, in embodiments in which the particles or particle chains are used to treat a lung condition, the particles or particle portions of particle chains delivered to the subject can have an arithmetic mean largest dimension of less than about 100 microns (e.g., less than about 50 microns). As another example, in embodiments in which the particles or particle chains are used to treat thyroid cancer, the particles or particle portions of particle chains can have a largest dimension of about 1,200 microns or less (e.g., from about 1,000 microns to about 1,200 microns). As an additional example, in some embodiments in which the particles are used only for therapeutic agent delivery, the particles can have an arithmetic mean maximum dimension of less than 100 microns (e.g., less than 50 microns, less than 10 microns, less than five microns).

The arithmetic mean maximum dimension of a group of particles can be determined using a Beckman Coulter RapidVUE Image Analyzer version 2.06 (Beckman Coulter, Miami, Fla.), described above. The arithmetic mean maximum dimension of a group of particles (e.g., in a composition) can be determined by dividing the sum of the diameters of all of the particles in the group by the number of particles in the group.

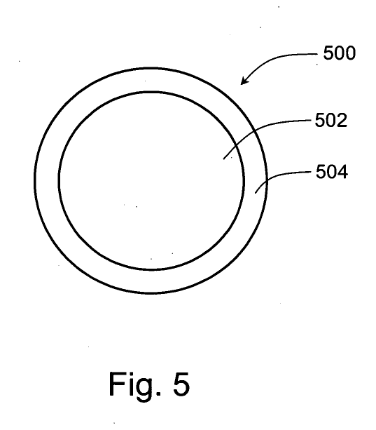

In certain embodiments, a particle or particle chain as described above can also include a coating. For example, FIG. 5 shows a particle 500 with an interior region 502 and a coating 504 formed of a polymer (e.g., polyvinyl alcohol) that is different from the polymer in in interior region 502. Coating 504 can, for example, regulate the release of therapeutic agent from particle 500, and/or can provide protection to interior region 502 of particle 500 (e.g., during delivery of particle 500 to a target site). In certain embodiments, coating 504 can be formed of a bioerodible and/or bioabsorbable material that can erode and/or be absorbed as particle 500 is delivered to a target site. This can, for example, allow interior region 502 to deliver a therapeutic agent to the target site once particle 500 has reached the target site. A bioerodible material can be, for example, a polysaccharide (e.g., alginate); a polysaccharide derivative; an inorganic, ionic salt; a water soluble polymer (e.g., polyvinyl alcohol, such as polyvinyl alcohol that has not been cross-linked); biodegradable poly DL-lactide-poly ethylene glycol (PELA); a hydrogel (e.g., polyacrylic acid, hyaluronic acid, gelatin, carboxymethyl cellulose); a polyethylene glycol (PEG); chitosan; a polyester (e.g., a polycaprolactone); a poly(ortho ester); a polyanhydride; a poly(lactic-co-glycolic) acid (e.g., a poly(d-lactic-co-glycolic) acid); a poly(lactic acid) (PLA); a poly(glycolic acid) (PGA); or a combination thereof. In some embodiments, coating 504 can be formed of a swellable material, such as a hydrogel (e.g., polyacrylamide co-acrylic acid). The swellable material can be made to swell by, for example, changes in pH, temperature, and/or salt. In certain embodiments in which particle 500 is used in an embolization procedure, coating 504 can swell at a target site, thereby enhancing occlusion of the target site by particle 500.

### Other Embodiments

While certain embodiments have been described, other embodiments are possible.

As an example, in some embodiments, a particle or particle chain can include a polymer and a bioabsorbable and/or bioerodible material dispersed uniformly or non-uniformly throughout the polymer. The bioabsorbable and/or bioerodible material can, for example, help to delay and/or moderate therapeutic agent release from the particle.

As a further example, in some embodiments, multiple cores could be included in the particles described above. In certain embodiments, the multiple cores could be formed of the same material of different materials.

As another example, in some embodiments in which a particle or particle chain that is used for embolization, the particle can also include one or more other embolic agents, such as a sclerosing agent (e.g., ethanol), a liquid embolic agent (e.g., n-butyl-cyanoacrylate), and/or a fibrin agent. The other embolic agent(s) can enhance the restriction of blood flow at a target site.

As an additional example, in some embodiments one or more particles or particle portions of particle chains is/are substantially nonspherical. In some embodiments, particles or particle portions of particle chains can be shaped during or after the particle or particle chain formation process to be nonspherical (e.g., ellipsoidal). In certain embodiments, particles or particle portions of particle chains can be shaped (e.g., molded, compressed, punched, and/or agglomerated with other particles) at different points in the manufacturing process. As an example, in some embodiments in which particles or particle chains include SIBS, the particles or particle portions of particle chains can be sufficiently flexible and/or moldable to be shaped. As another example, in certain embodiments in which particles or particle chains are formed using a gelling agent, the particles or particle chains can be physically deformed into a specific shape and/or size after the particles or particle chains have been contacted with the gelling agent, but before the polymer(s) in the particles or particle chains have been cross-linked. After shaping, the polymer(s) (e.g., polyvinyl alcohol) in the particles or particle chains can be cross-linked, optionally followed by substantial removal of gelling precursor (e.g., alginate). While substantially spherical particles or particle portions of particle chains have been described, in some embodiments, nonspherical particles or particle portions of particle chains can be manufactured and formed by controlling formation conditions. In some embodiments, nonspherical particles or particle portions of particle chains can be formed by post-processing the particles or particle portions of particle chains (e.g., by cutting into other shapes). Particle shaping is described, for example, in Baldwin et al., U.S. Patent Application Publication No. US 2003/0203985 A1, published on Oct. 30, 2003, and entitled “Forming a Chemically Cross-Linked Particle of a Desired Shape and Diameter,” which is incorporated herein by reference.

As a further example, in some embodiments, particles or particle chains can be used for tissue bulking. As an example, the particles or particle chains can be placed (e.g., injected) into tissue adjacent to a body passageway. The particles or particle chains can narrow the passageway, thereby providing bulk and allowing the tissue to constrict the passageway more easily. The particles or particle chains can be placed in the tissue according to a number of different methods, for example, percutaneously, laparoscopically, and/or through a catheter. In certain embodiments, a cavity can be formed in the tissue, and the particles or particle chains can be placed in the cavity. Particle tissue bulking can be used to treat, for example, intrinsic sphincteric deficiency (ISD), vesicoureteral reflux, gastroesophageal reflux disease (GERD), and/or vocal cord paralysis (e.g., to restore glottic competence in cases of paralytic dysphonia). In some embodiments, particle tissue bulking can be used to treat urinary incontinence and/or fecal incontinence. The particles or particle chains can be used as a graft material or a filler to fill and/or to smooth out soft tissue defects, such as for reconstructive or cosmetic applications (e.g., surgery). Examples of soft tissue defect applications include cleft lips, scars (e.g., depressed scars from chicken pox or acne scars), indentations resulting from liposuction, wrinkles (e.g., glabella frown wrinkles), and soft tissue augmentation of thin lips. Tissue bulking is described, for example, in Bourne et al, U.S. Patent Application Publication No. US 2003/0233150 A1, published on Dec. 18, 2003, and entitled “Tissue Treatment,” which is incorporated herein by reference.

As an additional example, in some embodiments, particles or particle chains can be used in an ablation procedure. For example, the particles or particle chains may include one or more ferromagnetic materials and may be used to enhance ablation at a target site. Ablation is described, for example, in Rioux et al., U.S. Patent Application Publication No. US 2004/0101564 A1, published on May 27, 2004, and entitled “Embolization;” Lanphere et al. U.S. Patent Application Publication No. US 2005/0129775 A1, published on Jun. 16, 2005, and entitled “Ferromagnetic Particles and Methods;” and Lanphere et al., U.S. patent application Ser. No. 11/117,156, filed on Apr. 28, 2005, and entitled “Tissue-Treatment Methods,” all of which are incorporated herein by reference.

As an additional example, in some embodiments, particles or particle chains having different shapes, sizes, physical properties, and/or chemical properties, can be used together in an embolization procedure. The different particles or particle chains can be delivered into the body of a subject in a predetermined sequence or simultaneously. In certain embodiments, mixtures of different particles or particle chains can be delivered using a multi-lumen catheter and/or syringe. In some embodiments, particles or particle chains having different shapes and/or sizes can be capable of interacting synergistically (e.g., by engaging or interlocking) to form a well-packed occlusion, thereby enhancing embolization. Particles or particle chains with different shapes, sizes, physical properties, and/or chemical properties, and methods of embolization using such particles are described, for example, in Bell et al., U.S. Patent Application Publication No. US 2004/0091543 A1, published on May 13, 2004, and entitled “Embolic Compositions,” and in DiCarlo et al., U.S. Patent Application Publication No. US 2005/0095428 A1, published on May 5, 2005, and entitled “Embolic Compositions,” both of which are incorporated herein by reference.

Other embodiments are in the claims.

1. A particle comprising:
one or more polymer fiber strands collectively organized into the overall shape of a particle, the fiber strands being distributed such that voids are formed within the particle.

2. A particle as defined in claim 1, wherein the largest dimension of the particle is at most 5,000 microns.

3. A particle as defined in claim 1, wherein the particle is spherical.

4. A particle as defined in claim 1, wherein the polymer fiber strands are randomly oriented.

5. A particle as defined in claim 1, wherein the polymer fiber strands comprise SIBS.

6. A particle as defined in claim 1, wherein the polymer fiber strands are elastic.

7. A particle as defined in claim 1, wherein the particles are compressible.

8. A particle as defined in claim 1, further comprising a therapeutic agent.

9. A particle as defined in claim 1, further comprising a core.

10. A particle as defined in claim 9, wherein the one or more polymer fiber strands are wound around the core.

11. A particle as defined in claim 9, wherein the core comprises a different material than the fiber strands.

12. A particle as defined in claim 11, wherein the core comprises a metal.

13. A particle as defined in claim 12, wherein the metal is electrically charged.

14. A particle as defined in claim 9, wherein the core comprises a polymer.

15. A particle as defined in claim 14, wherein the polymer is electrically charged.

16. A particle as defined in claim 9, wherein the core comprises a radioactive material.

17. A particle as defined in claim 9, wherein the core is hollow.

18. A particle as defined in claim 1, wherein the voids are accessible from the exterior of the particle.

19. A particle as defined in claim 1, wherein each fiber strand is physically bonded at one or more points along its length to an overlapping intra-strand or inter-strand segment.

20. A particle chain comprising a particle as defined in claim 1 connected by a link to at least one other particle.

21. A particle chain as defined in claim 20, wherein the at least one other particle is a particle as defined in claim 1.

22. A particle chain as defined in claim 20, wherein the link is a polymer.

23. A particle chain as defined in claim 20, wherein the link is a metal.

24. A particle chain as defined in claim 20, wherein the link is a fiber.

25.-51. (canceled)
